(19) United States
(12) Patent Application Publication
(10) Pub. No.: US 2024/0026312 A1
(43) Pub. Date: Jan. 25, 2024

(54) PURIFICATION OF PROTEINS WITH CATIONIC SURFACTANT

(71) Applicant: Horizon Therapeutics USA, Inc., Deerfield, IL (US)

(72) Inventors: Meir FISCHER, Rehovot (IL); Eliyahu HAROSH, Ashdod (IL)

(21) Appl. No.: 18/376,684

(22) Filed: Oct. 4, 2023

Related U.S. Application Data
(63) Continuation of application No. 17/325,555, filed on May 20, 2021, now abandoned.
Application No. 17/325,555 is a continuation of application No. 16/455,073, filed on Jun. 27, 2019, now abandoned.
Application No. 16/455,073 is a division of application No. 15/356,046, filed on Nov. 18, 2016, now abandoned.
Application No. 15/356,046 is a division of application No. 11/918,292, filed on Aug. 14, 2009, now Pat. No. 9,534,013.

(51) Int. Cl. C12N 9/06; C07K 1/30; C07K 14/565; C07K 16/00; C07K 16/36; C12P 21/02

(52) U.S. Cl. CPC C12N 9/0048; C07K 1/30; C07K 14/565; C07K 16/00; C07K 16/36; C12P 21/02; C12Y 107/03003; C07K 2317/622; C07K 2317/76

(57) ABSTRACT

The subject invention provides a method for purifying a target protein from a mixture comprising the target protein and contaminating protein, comprising the steps of exposing the mixture to an effective amount of a cationic surfactant such that the contaminating protein is preferentially precipitated and recovering the target protein. Proteins purified according to the method of the invention are also provided.

### CROSS-REFERENCE TO RELATED APPLICATIONS

The present application is a continuation of U.S. application Ser. No. 17/325,555, filed May 20, 2021, which is a continuation of U.S. application Ser. No. 16/455,073, filed Jun. 27, 2019, which is a division of U.S. application Ser. No. 15/356,046, filed Nov. 18, 2016, which is a division of U.S. application Ser. No. 11/918,292, filed Aug. 14, 2009, now U.S. Pat. No. 9,534,013, which is a U.S. National Phase application under 35 U.S.C. § 371 of International Application No. PCT/US2006/013751, filed Apr. 12, 2006, which claims priority to and the benefit of U.S. provisional application Ser. No. 60/670,520, filed Apr. 11, 2005, the disclosures of which are being incorporated by reference herein in their entireties.

### FILED OF INVENTION

The invention relates to the field of protein purification using surfactants.

### BACKGROUND

Production of biological macromolecules, particularly proteins, often involves purity-enhancing steps based on physical and physicochemical properties. Difficulties encountered in such process steps include, but are not limited to, determining conditions which enable separation of soluble and insoluble molecules, relatively low recovery of the desired molecule after a treatment step, loss of biological activity in the course of the process, and sensitivity of the protein to process step conditions such as pH.

Surfactants have been utilized in the processing of biological macromolecules. Cationic surfactants are a recognized subclass of surfactants, and include amphipathic ammonium compounds. Amphipathic ammonium compounds comprise quaternary ammonium compounds of the general formula QN⁺ and paraffin chain primary ammonium compounds of the general formula RNH₃⁺. Both types of amphipathic ammonium compounds include long-chain ammonium surfactants that have a long aliphatic chain of preferably at least six carbon atoms (Scott (1960) Methods Biochem. Anal. 8:145-197, incorporated herein by reference in its entirety). The long-chain quaternary ammonium surfactants are known to interact with biological macromolecules. The long-chain quaternary ammonium compounds have at least one substituent at the nitrogen which consists of a linear alkyl chain with 6-20 carbon atoms. The best known representatives of this class are the benzalkonium salts (chlorides and bromides), hexadecylpyridinium chloride dequalinium acetate, cetyldimethylammonium bromide (CTAB) and hexadecylpyridinium chloride (CPCI), and benzethonium chloride. Quaternary ammonium surfactants include salts such as cetyl pyridinium salts, e.g. cetyl pyridinium chloride (CPC), stearamide-methylpyridinium salts, lauryl pyridinium salts, cetyl quinolynium salts, lauryl aminopropionic acid methyl ester salts, lauryl amino propionic acid metal salts, lauryl dimethyl betaine stearyl dimethyl betaine, lauryl dihydroxyethyl betaine and benzethonium salts. Alkyl pyridinium salts comprise stearyl-trimethyl ammonium salts, alkyl-dimethylbenzyl-ammonium chloride, and dichloro-benzyldimethyl-alkylammonium chloride.

Known uses of cationic surfactants for purifying biological macromolecules include 1) solubilization of aggregates, including protein aggregates; 2) elution of chromatographic column-bound biological macromolecules; and 3) precipitation of polyanions such as hyaluronic acid (HA), nucleic acids, and heparin (and molecules which co-precipitate with polyanions).

Cationic surfactants have been used for solubilizing protein aggregates. Otta and Bertini ((1975) Acta Physiol. Latinoam. 25:451-457, incorporated herein by reference in its entirety) demonstrated that active uricase could be solubilized from rodent liver peroxizomes with the quaternary ammonium surfactant, Hyamine 2389. It is found that increase of the ammonium surfactant concentration resulted in increase of dissolution of both uricase (based on enzymatic activity) and total protein such that there is no increase in the relative amount of uricase protein with respect to the amount of total protein. In other words, there was no selective solubilization of the uricase protein with respect to the total protein, and the uricase protein did not constitute a higher percentage of the total protein upon solubilization with the cationic surfactant. Thus, in this process, uricase purity with respect to the total protein content is apparently not enhanced as a result of quaternary ammonium surfactant solubilization.

In another study, Truscoe ((1967) Enzymologia 33:1 19-32, incorporated herein by reference in its entirety) examined a panel of cationic, anionic, and neutral detergents for their extraction efficacy of urate oxidase (uricase) from ox kidney powders. While the neutral and anionic detergents were found to enhance soluble urate oxidase activity, the cationic detergents, e.g., quaternary ammonium salts, were found to decrease total enzymatic activity with increasing concentration. The authors concluded that cationic detergents were not useful for purifying ox kidney urate oxidase.

Solubilization of recombinant proteins, porcine growth hormone, methionyl-porcine growth hormone, infectious bursal disease virus protein, B-galactosidase fusion protein, from E. coli inclusion bodies or cells, with cationic surfactants is described in U.S. Pat. Nos. 4,797,474, 4,992,531, 4,966,963, and 5,008,377, each incorporated herein by reference in its entirety. Solubilization under alkaline conditions is accomplished using quaternary ammonium compounds including cetyltrimethylammonium chloride, mixed n-alkyl dimethyl benzylammonium chloride, CPC, N,N-dimethyl-N-[2-[2-[4-(1,1,3,3,-tetramethylbutyl)-phenoxy]ethoxy]ethyl]benzenemethanammonium chloride, tetradecyl trimethylammonium bromide, dodecyl trimethylammonium bromide, cetyl trimethylammonium bromide. These publications mention that, after each solubilization process, the solutions are centrifuged, and little to no pellet is observed in each case. This observation suggests that most or all of the proteins are solubilized without regard to selectivity for the solubilization of a target protein. The purity of the recovered proteins is not indicated. U.S. Pat. No. 5,929,231, incorporated herein by reference in its entirety, describes cetyl pyridinium chloride (CPC) disintegration of granules and aggregates containing starches. Thus, the prior art relates to use of cationic surfactants for general, nonspecific solubilization of particulate biological macromolecules. These methods of the prior art do not disclose increasing the purity of a desired target protein with respect to total protein with a cationic surfactant.

Cationic surfactants have also been used to elute biological macromolecules adsorbed to cation exchange resins or aluminum-containing adjuvants (Antonopoulos, et al. (1961) Biochim. Biophys. Acta 54:213-226; Embery (1976) J. Biol. Buccale 4:229-236; and Rinella, et al. (1998) J. Colloid Interface Sci. 197:48-56, each of which is incorporated herein by reference in its entirety). U.S. Pat. No. 4,169,764, incorporated herein by reference in its entirety, describes elution of urokinase from carboxymethyl cellulose columns using a wide variety of cationic surfactant solutions. The authors state a preference for using tetra substituted ammonium salts in which one alkyl group is a higher alkyl group up to 20 carbon atoms and the others are lower alkyl groups up to 6 carbon atoms. Use of such cationic surfactants enables removal of biological macromolecules from their attachment to a solid matrix.

Conversely, impregnation of filters such as those composed of nylon, with cationic surfactant enables immobilizing of polysaccharides or nucleic acids (Maccari and Volpi (2002) Electrophoresis 23:3270-3277; Benitz, et al. (1990) U.S. Pat. No. 4,945,086; Macfarlane (1991) U.S. Pat. No. 5,010,183, each of which is incorporated herein by reference in its entirety). This phenomenon is apparently due to cationic surfactant-polyanion interactions which enable precipitation of the polyanion.

It is well established that amphipathic ammonium compounds, which comprise quaternary ammonium compounds of the general formula QN⁺ and paraffin chain primary ammonium compounds of the general formula RNH₃⁺, can precipitate polyanions under defined conditions (reviewed in Scott (1955) Biochim. Biophys. Acta 18:428-429; Scott (1960) Methods Biochem. Anal. 8:145-197; Laurent, et al., (1960) Biochim. Biophys. Acta 42:476-485; Scott (1961) Biochem. J. 81:418-424; Pearce and Mathieson (1967) Can. J. Biochemistry 45:1565-1576; Lee (1973) Fukushima J. Med. Sci. 19:33-39; Balazs, (1979) U.S. Pat. No. 4,141,973; Takemoto, et al., (1982) U.S. Pat. No. 4,312,979; Rosenberg (1981) U.S. Pat. No. 4,301,153; Takemoto, et al., (1984) U.S. Pat. No. 4,425,431; d'Hinterland, et al., (1984) U.S. Pat. No. 4,460,575; Kozma, et al. (2000) Mol. Cell. Biochem. 203:103-112, each of which is incorporated herein by reference in its entirety). This precipitation is dependent on the precipitating species having a high polyanion charge density and high molecular weight (Saito (1955) Kolloid-Z 143:66, incorporated herein by reference in its entirety). The presence of salts can interfere with or reverse cationic surfactant-induced precipitation of polyanions.

Additionally, polyanions can be differentially precipitated from solutions containing protein contaminants, under alkaline pH conditions. In such cases, proteins not chemically bound to the polyanions will remain in solution, while the polyanions and other molecules bound to the polyanions will precipitate. For example, precipitation of polyanions such as polysaccharides and nucleic acids is accompanied by co-precipitation of molecules such as proteoglycans and proteins interacting with the polyanions (Blumberg and Ogston (1958) Biochem. J. 68:183-188; Matsumura, et al., (1963) Biochim. Biophys. Acta 69: 574-576; Serafini-Fracassini, et al. (1967) Biochem. J. 105:569-575; Smith, et al. (1984) J. Biol. Chem. 259:11046-11051; Fuks and Vlodavsky (1994) U.S. Pat. No. 5,362,641; Hascall and Heinegard (1974) J. Biol. Chem. 249:4232-4241, 4242-4249, and 4250-4256; Heinegard and Hascall (1974) Arch. Biochem. Biophys. 165: 427-441; Moreno, et al. (1988) U.S. Pat. No. 4,753,796; Lee, et al. (1992) J. Cell Biol. 116: 545-557; Varelas, et al. (1995) Arch. Biochem. Biophys. 321: 21-30, each of which is incorporated herein by reference in its entirety).

The isoelectric point (or pI) of a protein is the pH at which the protein has an equal number of positive and negative charges. Under solution conditions with pH values close to (especially below) a protein's isoelectric point, proteins can form stable salts with strongly acidic polyanions such as heparin. Under conditions which promote precipitation of such polyanions, the proteins complexed with the polyanions also precipitate (LB Jaques (1943) Biochem. J. 37:189-195; A S Jones (1953) Biochim. Biophys. Acta 10:607-612; JE Scott (1955) Chem and Ind 168-169; U.S. Pat. No. 3,931,399 (Bohn, et al., 1976) and U.S. Pat. No. 4,297,344 (Schwinn, et al., 1981), each of which is incorporated herein by reference in its entirety).

U.S. Pat. Nos. 4,421,650, 5,633,227, and Smith, et al. ((1984) J. Biol. Chem. 259:11046-11051, each of which is incorporated herein by reference in its entirety) describe purification of polyanions by sequential treatment with a cationic surfactant and ammonium sulfate (that enables dissociation of polyanion-cationic surfactant complexes) and subsequent separation using hydrophobic interactions chromatography. European patent publication EP055188, incorporated herein by reference in its entirety, describes cationic surfactant-enabled separation of RTX toxin from lipopolysaccharide. However, there is no mass balance in the amount of lipopolysaccharide that is quantified by endotoxin activity assays. Neutralization of endotoxin activity by strongly interacting cationic compounds has been demonstrated (Cooper J F (1990) J Parenter Sci Technol 44:13-5, incorporated herein by reference in its entirety). Thus, in EP055188, the lack of endotoxin activity in the precipitate following treatment with increasing amounts of cationic surfactant possibly results from neutralization of the activity by surfactant-lipopolysaccharide complex formation.

The above-mentioned methods require intermediary polyanions, solid supports or aggregates comprising proteins with selective solubility by a cationic surfactant for enabling purification of soluble proteins using cationic surfactant. Hence, the prior art does not provide a method of purifying a target protein by contacting the protein with a cationic surfactant in an amount effective to preferentially precipitate proteins other than the target protein, i.e., contaminating proteins, particularly when such contacting is done in the absence of intermediary polyanions, solid supports, or aggregates of proteins. Often, one skilled in the art encounters mixtures of soluble proteins and does not have a simple, efficient means for purifying the desired protein. The novel method for purifying proteins, described herein, enables efficient purification of target proteins by using cationic surfactants to preferentially precipitate proteins other than the target protein. Preferably such precipitation of contaminating proteins is direct, and does not depend upon the presence of polyanions, solid supports or aggregates comprising the contaminating proteins and other molecules.

### SUMMARY OF THE INVENTION

The subject invention provides a method for purifying a target protein from a mixture comprising the target protein and contaminating protein, comprising the steps of exposing the mixture to an effective amount of a cationic surfactant such that the contaminating protein is preferentially precipitated and recovering the target protein.

### BRIEF DESCRIPTION OF THE FIGURES

FIG. 1A through FIG. 1C depict the effects of CPC concentration on the activity and purity of mammalian uricase from dissolved E. coli inclusion bodies, which are measured following the indicated CPC treatments and centrifugal separation.

FIG. 1A shows protein concentration of mammalian uricase.

FIG. 1B shows enzymatic activity of mammalian uricase.

FIG. 1C shows the specific activity of each isolate calculated as a ratio of these values (activity/protein concentration).

FIG. 2A through FIG. 2B depict size-exclusion HPLC chromatographic analysis of crude mammalian uricase prepared from inclusion bodies and following treatment with 0.075% CPC. The areas of each peak and the percent of total area are summarized in the adjacent tables.

FIG. 2A shows size-exclusion HPLC profiles of solubilized E coli inclusion bodies without CPC treatment.

FIG. 2B shows the supernatant following CPC (0.075%) precipitation and filtration.

FIG. 3 depicts SDS-PAGE (15% gel) analysis of CPC treated uricase.

The uricase-containing samples are prepared as described in Example 1. Samples from various process steps are aliquoted as follows: Lane 1—dissolved IBs; Lane 2—supernatant after CPC treatment; Lane 3—pellet after CPC treatment.

FIG. 4A through FIG. 4C depict size-exclusion HPLC analysis of crude scFv antibody following treatment with 0.02% CPC. The areas of each peak and the percent of total area are summarized in the adjacent tables.

FIG. 4A shows size-exclusion HPLC profiles.

FIG. 4B shows reference standard BTG-271 scFv antibody and solubilized inclusion bodies.

FIG. 4C shows the supernatant following refolding and CPC (0.02%) precipitation and filtration are analyzed.

FIG. 5 depicts SDS-PAGE (15% gel) analysis of CPC treated scFv antibody.

The scFv antibody-containing samples from various process steps and standards are presented in the following order: Lane 1—molecular weight standards; Lane 2—dissolved IBs; Lane 3—refolded protein; Lane 4—CPC pellet; Lane 5—supernatant after CPC treatment.

FIG. 6A through FIG. 6B depict HPLC gel filtration chromatography of interferon beta before and after treatment with CPC.

FIG. 6A. Before CPC treatment.

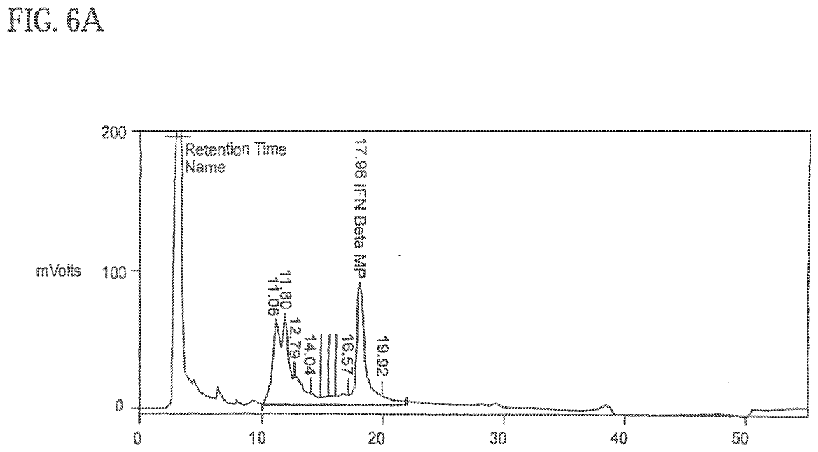

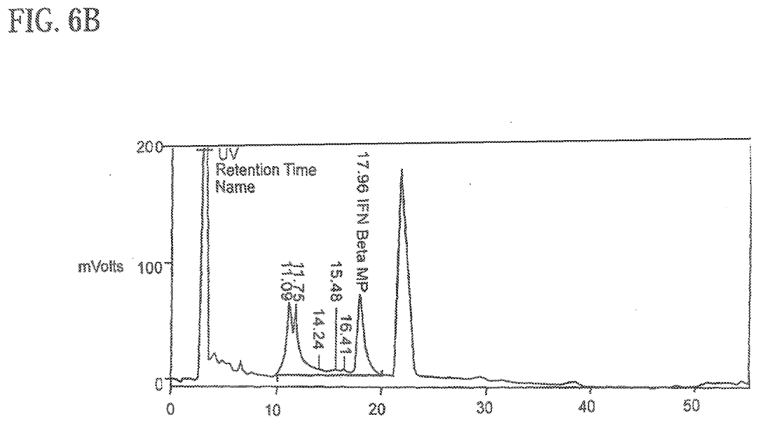

FIG. 6B. After CPC treatment.
200 μl of a solution of 0.1 mg/ml interferon beta was loaded into the column.

### DETAILED DESCRIPTION OF THE INVENTION

Proteins are ampholytes, having both positive and negative charges. The pH of a solution and charged molecules that interact with a protein impact the net charge of that protein. Strong interactions between proteins can occur when the net charge of a protein is neutral (the isoelectric point). When the pH of the solution is below the isoelectric point of the protein, the protein has a net positive charge, and there may be electrostatic repulsion between cationic molecules, including other proteins.

It is an object of the invention to provide a method for purifying a solubilized target protein from a solution comprising a mixture of the target protein and contaminating proteins comprising contacting the solubilized mixture with an effective amount of a cationic surfactant and recovering the target protein. Cationic surfactants are surface-active molecules with a positive charge. In general, these compounds also have at least one non-polar aliphatic group. Preferably the target protein has an isoelectric point greater than 7. In a particular embodiment, the pH of the solution is about the same as the isoelectric point of the target protein. In a preferred embodiment, the pH of the solution is less than the isoelectric point of the target protein. In a particular embodiment, when the pH of the solution is above the isoelectric point of the target protein, the pH of the solution is within 1-2 pH units of the isoelectric point of the target protein. In a particular embodiment, when the pH of the solution is above the isoelectric point of the target protein, the pH of the solution is within 1 pH unit of the isoelectric point of the target protein.

In a particular embodiment, the contaminating protein or proteins are preferentially precipitated, thereby increasing the proportion of the proteins remaining in solution represented by the target protein. For example, starting from a solution of target protein and contaminating protein wherein the target protein is 20% of the total protein in solution, one can purify the target protein using the methods provided to achieve a solution wherein the target protein is 30% or more, 40% or more, 50% or more, 60% or more, 70% or more, 80% or more, 95% or more, 95% or more of the total protein remaining in solution.

As used herein, the term “preferentially precipitate” means that a protein or group of proteins are precipitated to a greater extent than another protein or group of proteins. For example, in the case of a mixture of a target protein and contaminating proteins, the contaminating proteins are preferentially precipitated with respect to the target protein when 20% or more of the contaminating proteins are precipitated, while less than 20% of the target protein is precipitated. Preferably, a high percentage of contaminating proteins are precipitated, while a low percentage of the target protein is precipitated. In preferred embodiments, 30% or more of the contaminating proteins are precipitated, while less than 30% of the target protein is precipitated; 40% or more of the contaminating proteins are precipitated, while less than 40% of the target protein is precipitated; 50% or more of the contaminating proteins are precipitated, while less than 50% of the target protein is precipitated: 60% or more of the contaminating proteins are precipitated, while less than 60% of the target protein is precipitated; 70% or more of the contaminating proteins are precipitated, while less than 70% of the target protein is precipitated; 80% or more of the contaminating proteins are precipitated, while less than 80% of the target protein is precipitated: 90% or more of the contaminating proteins are precipitated, while less than 90% of the target protein is precipitated; 95% or more of the contaminating proteins are precipitated, while less than 95% of the target protein is precipitated. Preferably, a small percentage of the target protein is precipitated. For example, less than 60%, less than 50%, less than 40%, less than 30%, less than 20%, less than 10%, less than 5% or less than 1% of the target protein is precipitated.

In a particular embodiment, the total amount of protein in solution (target protein plus contaminating protein), prior to carrying out the purification method of the invention, is from 0.1 to 10 mg/ml. In particular embodiments, the total amount of protein in solution prior to carrying out the purification method of the invention is from 0.1 to 3 mg/ml, 0.3 to 2 mg/ml, 0.5 to 2 mg/ml, 0.5 to 1 mg/ml, 1 to 2 mg/ml, or about 1 mg/ml.

In particular embodiments, the preferential precipitation of contaminating proteins is direct, and does not depend, or does not substantially depend, upon the presence of polyanions. In another embodiment, the preferential precipitation of contaminating proteins is direct, and does not depend, or does not substantially depend, upon the presence of a solid support. In another embodiment, the preferential precipitation of contaminating proteins does not depend, or does not substantially depend, upon the presence of aggregates between contaminating proteins and other molecules. The preferential precipitation of contaminating proteins does not depend or substantially depend upon a component (e.g., polyanions, solid supports, or aggregates of contaminating proteins and other molecules) when, for example, the removal of that component does not effect or does not substantially effect, respectively, the preferential precipitation of contaminating protein. An example of an insubstantial effect of the removal of a component would be that the contaminating proteins are preferentially precipitated both when the component is present and when it is absent. A further example would be the contaminating proteins are preferentially precipitated to the same extent when the component is present and when it is absent. Preferably, the same or substantially the same amount of contaminating proteins are precipitated in the absence or substantial absence of the component as is in the presence of the component.

In another embodiment, the method is performed in the absence of polyanions or in the absence of substantial amounts of polyanions. In another embodiment, the method is performed in the absence of a solid support or in the absence of a substantial solid support. In another embodiment, the method is performed in the absence of aggregates between contaminating proteins and other molecules, or in the absence of substantial amounts of aggregates between contaminating proteins and other molecules. Preferably, the method is performed in the absence of or in the absence of substantial amounts of two or three members of the group consisting of polyanions; a solid support; and aggregates between contaminating proteins and other molecules.

Once provided the method of the invention, it is routine for one of skill in the art to select the particular surfactant used and the conditions, e.g., pH, temperature, salinity, cationic surfactant concentration, total protein concentration, under which this procedure is accomplished to enhance efficiency of the purification of a particular target protein. For example, purifications performed at differing pH values and surfactant concentrations may be compared to establish the optimal purification conditions. Examples of this procedure are provided below in the Examples section. In a particular embodiment, the pH of the solution is chosen such that it is as high as is possible without substantially reducing the amount of target protein recovered.

It is a further objective of the invention to provide a method for determining conditions which enable efficient purification of target proteins on the basis of their solubility, as impacted by cationic surfactants.

An effective amount of cationic surfactant is an amount of surfactant that causes the preferential precipitation of contaminating proteins. In particular embodiments, the effective amount of surfactant precipitates 40%, 50%, 60%, 70%, 80%, 90°/%, 95% or 99% of the contaminating proteins.

In an embodiment of the invention, the cationic surfactant is added to a concentration of from 0.001% to 5.0%, preferably the cationic surfactant is added to a concentration of from 0.01% to 0.5% and more preferably, the cationic surfactant is added to a concentration of from 0.03% to 0.2%. In particular embodiments, the cationic surfactant is added to a concentration of from 0.01% to 0.1%, 0.01% to 0.075%, 0.01% to 0.05% or 0.01% to 0.03%.

In an embodiment of the invention, the above-mentioned method is accomplished when the cationic surfactant is an amphipathic ammonium compound.

In a preferred embodiment, the solubilized target protein is subjected to further processing after contaminating proteins have been preferentially precipitated. Such further processing can include additional purification steps, assays for activity or concentration, dialysis, chromatography (e.g., HPLC, size exclusion chromatography), electrophoresis, dialysis, etc.

As used herein, amphipathic ammonium compounds comprise compounds having both cationic and non-polar components with the general formula of either QN⁺ or RNH₃⁺. Q indicates that the nitrogen is a quaternary ammonium (covalently bonded to four organic groups which may or may not be bonded one to another). When organic groups are bonded one to another, they may form cyclic aliphatic or aromatic compounds, depending on the electronic configuration of the bonds between the components which form the cyclic structure. When the amphipathic ammonium compound selected has the general formula, RNH₃⁺, the compound is a primary amine wherein R is an aliphatic group. Aliphatic groups are open chain organic groups.

In an embodiment of the invention, the selected amphipathic ammonium compound may form a salt with a halide. Commonly, halide salts refer to those comprising fluoride, chloride, bromide, and iodide ions.

In an embodiment of the invention, the amphipathic ammonium compound has at least one aliphatic chain having 6-20 carbon atoms, preferably, the amphipathic ammonium compound has at least one aliphatic chain having 8-18 carbon atoms.

In an embodiment of the invention, the selected amphipathic ammonium compound is selected from the group consisting of cetyl pyridinium salts, stearamide-methylpyridinium salts, lauryl pyridinium salts, cetyl quinolynium salts, lauryl aminopropionic acid methyl ester salts, lauryl amino propionic acid metal salts, lauryl dimethyl betaine, stearyl dimethyl betaine, lauryl dihydroxyethyl betaine and benzethonium salts.

Amphipathic ammonium compounds which may be used include, but are not limited to hexadecylpyridinium chloride dequalinium acetate, hexadecylpyridinium chloride, cetyltrimethylammonium chloride, mixed n-alkyl dimethyl benzylammonium chloride, cetyl pyridinium chloride (CPC), N,N-dimethyl-N-[2-[2-[4-(1,1,3,3,-tetramethylbutyl)-phenoxy]ethoxy]ethyl] benzenemethanammonium chloride, alkyl-dimethylbenzyl-ammonium chloride, and dichloro-benzyldimethyl-alkylammonium chloride, tetradecyl trimethylammonium bromide, dodecyl trimethylammonium bromide, cetyl trimethylammonium bromide, lauryl dimethyl betaine stearyl dimethyl betaine, and lauryl dihydroxyethyl betaine.

In an embodiment of the invention, the amphipathic ammonium compound is a cetylpyridinium salt such as cetylpyridinium chloride.

In an embodiment of the invention, the mixture containing the desired protein further comprises cellular components such as cellular components derived from microorganisms, for example, bacteria such as E. coli.

In an embodiment of the invention, the cellular components are one or more proteins.

In an embodiment of the invention the target protein may be a recombinant protein, for example, an enzyme.

The method of the invention can be used to purify a variety of proteins. These proteins may include, but are not limited to antibodies, uricase, interferon-beta, leech factor X inhibitor, acid deoxyribonuclease II, elastase, lysozyme, papain, peroxidase, pancreatic ribonuclease, trypsinogen, trypsin, cytochrome c, erabutoxin, Staphylococcus aureus enterotoxin Cl, and monoamine oxidase A, and other proteins that are positively charged under alkaline conditions.

In an embodiment of the invention the target protein may be an antibody, receptor, enzyme, transport protein, hormone, or fragment thereof or a conjugate e.g., conjugated to a second protein or a chemical or a toxin.

Antibodies include but are not limited to monoclonal, humanized, chimeric, single chain, bispecific, Fab fragments, F(ab′)2 fragments, fragments produced by a Fab expression library, anti-idiotypic (anti-Id) antibodies, and epitope-binding fragments of any of the above, but with the proviso that at the conditions of the purification the antibody is positively charged.

For preparation of monoclonal antibodies, any technique that provides for the production of antibody molecules by continuous culture of cell lines may be used. These include but are not limited to the hybridoma technique of Kohler and Milstein, (1975, Nature 256, 495-497; and U.S. Pat. No. 4,376,110), the human B-cell hybridoma technique (Kozbor et al., 1983, Immunology Today 4, 72; Cole et al., 1983, Proc. Natl. Acad. Sci. USA 80, 2026-2030), and the EBV-hybridoma technique to produce human monoclonal antibodies (Cole et al., 1985, Monoclonal Antibodies And Cancer Therapy, Alan R. Liss, Inc., pp. 77-96).

Such antibodies may be used as the basis from which to clone and thus recombinantly express individual heavy and light chains. The two chains may be recombinantly expressed in the same cell or combined in vitro after separate expression and purification. Nucleic acids (e.g., on a plasmid vector) encoding a desired heavy or light chain or encoding a molecule comprising a desired heavy or light chain variable domain can be transfected into a cell expressing a distinct antibody heavy or light chain or molecule comprising an antibody heavy or light chain, for expression of a multimeric protein. Alternatively, heavy chains or molecules comprising the variable region thereof or a CDR thereof can optionally be expressed and used without the presence of a complementary light chain or light chain variable region. In other embodiments, such antibodies and proteins can be N or C-terminal modified, e.g., by C-terminal amidation or N-terminal acetylation.

A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 5,816,397.) Techniques for the production of chimeric antibodies include the splicing the genes from a mouse antibody molecule of appropriate antigen specificity together with genes from a human antibody molecule of appropriate biological activity (see for example, Morrison, et al., 1984, Proc. Natl. Acad. Sci., 81, 6851-6855; Neuberger, et al., 1984, Nature 312, 604-608; Takeda, et al., 1985, Nature 314, 452-454).

Humanized antibodies are antibody molecules from non-human species having one or more complementarity-determining regions (CDRs) from the non-human species and framework regions from a human immunoglobulin molecule. Techniques for the production of humanized antibodies are described for example in Queen, U.S. Pat. No. 5,585,089 and Winter, U.S. Pat. No. 5,225,539. The extent of the framework regions and CDRs have been precisely defined (see, “Sequences of Proteins of Immunological Interest”, Kabat, E. et al., U.S. Department of Health and Human Services (1983).

Single chain antibodies are formed by linking the heavy and light chain fragments of the Fv region via an amino acid bridge, resulting in a single chain polypeptide. Techniques for the production of single chain antibodies are described for example in U.S. Pat. No. 4,946,778; Bird, 1988, Science 242, 423-426; Huston, et al., 1988, Proc. Natl. Acad. Sci. USA 85, 5879-5883; and Ward, et al., 1989, Nature 334, 544-546).

A bispecific antibody is a genetically engineered antibody which recognizes two types of targets e.g. (1) a specific epitope and (2) a “trigger” molecule e.g. Fc receptors on myeloid cells. Such bispecific antibodies can be prepared either by chemical conjugation, hybridoma, or recombinant molecular biology techniques.

Antibody fragments include but are not limited to: The F(ab′)2 fragments, which can be produced by pepsin digestion of the antibody molecule and the F(ab′) fragments, which can be generated by reducing the disulfide bridges of the F(ab′)2 fragments. Alternatively, Fab expression libraries may be constructed (Huse, et al., 1989, Science 246, 1275-1281) to allow rapid and easy identification of monoclonal Fab fragments with the desired specificity.

In an embodiment of the invention, the protein is uricase.

In another embodiment of the invention, the uricase is a mammalian uricase.

In another embodiment of the invention, the mammalian uricase is a variant mammalian uricase.

In another embodiment of the invention, the mammalian uricase is a porcine uricase.

In another embodiment of the invention, the variant porcine uricase is designated PKSAN uricase.

In another embodiment of the invention, the protein is an antibody.

In another embodiment of the invention, the antibody is a single chain antibody.

In another embodiment of the invention, the protein is an interferon.

In another embodiment of the invention the interferon is interferon beta. In a particular embodiment, the interferon is interferon beta 1b. Nagola, S. et al., Nature, 284:316 (1980); Goeddel, D. V. et al., Nature, 287:411 (1980); Yelverton, E. et al., Nuc. Acid Res., 9:731 (1981); Streuli, M. et al., Proc. Nat'l Acad. Sci. (U.S.), 78:2848 (1981); European Pat. Application No. 28033, published May 6, 1981; 321134, published Jul. 15, 1981; 34307 published Aug. 26, 1981; and Belgian Patent No. 837379, issued Jul. 1, 1981 described various methods for the production of beta-interferon employing recombinant DNA techniques. Procedures for recovering and purifying bacterially produced IFNs are described in U.S. Pat. Nos. 4,450,103; 4,315,852; 4,343,735; and 4,343,736; and Derynck et al., Nature (1980) 287:193-197 and Scandella and Kornberg, Biochemistry, 10:4447 (1971).

In a particular embodiment, the target protein is leech factor Xa. Leech factor Xa may be produced by any method known to one of skill in the art, such as the method described in U.S. Pat. No. 6,211,341 and International Patent Publication No. WO94/23735.

In an embodiment of the invention, the contacting is done for between about 1 minute and about 48 hours, more preferably from about 10 minutes to about 24 hours, about 30 minutes to about 12 hours, about 30 minutes to about 8 hours, about 30 minutes to about 6 hours, about 30 minutes to about 4 hours, about 30 minutes to about 2 hours, about 30 minutes to about 1 hour, or about 1 to about 2 hours.

In an embodiment of the invention, the contacting is done at a temperature from about 4° C. to about 36° C.; more preferably from about 4° C. to about 26° C.

The subject invention also provides use of cationic surfactant as a single agent for purifying a protein having an isoelectric point greater than 7 under alkaline conditions.

The subject invention also provides a uricase purified under alkaline conditions from a mixture by the addition of cetylpyridinium chloride to the mixture.

In an embodiment of the invention, the uricase is obtained from a bacterial cell comprising DNA encoding the uricase by a method comprising treating the bacterial cell so as to express the DNA and produce the uricase and recovering the uricase.

In an embodiment of the invention, the uricase is recovered from precipitates within the bacterial cell.

The subject invention also provides purified uricase for use in preparing a uricase-polymer conjugate.

The invention also provides a purified protein having an isoelectric point greater than 7 obtainable by a method comprising contacting a mixture containing the protein with an effective amount of a cationic surfactant under conditions such that the protein is positively charged or has an area of positive charge, and recovering the protein.

The subject invention also provides use of a cetylpyridinium salt for purifying a protein having an isoelectric point greater than 7.

As to the pH, in embodiments where the mixture is contacted with an effective amount of a cationic surfactant under conditions such that the target protein is positively charged, the pH will vary with the nature of the target protein. However, the pH is preferably between pH7 and pH11; preferred ranges are from about pH7 and pH10, pH7 to pH9, pH8 to pH1, pH8 to pH10 or pH8 to pH9.

### EXAMPLES

The examples which follow are set forth to aid in understanding the invention but are not intended to and should not be construed to limit its scope in any way.

### Example 1. Use of CPC for Purification of Recombinant Mammalian Uricase

### 1.1. Background

Pharmaceutical grade uricase must be essentially free of non-uricase protein. Mammalian uricase (isoelectric point of 8.67) produced in E. coli accumulated intracellularly in precipitates similar to organelles referred to as inclusion bodies (IBs) which can be easily isolated for further purification. In contrast to the classical view that IBs contain scrambled/mis-folded expressed protein, these IB-like elements contain correctly folded uricase in a precipitated form. Exposure of uricase IB-like elements to an alkaline pH, e.g., about pH 9-11, re-dissolved the precipitated protein. The uricase content in solubilized IB-like elements was about 40-60% and required extensive purification to obtain a homogeneous uricase preparation. Herein, we demonstrate purification of uricase and other protein with CPC that can be assessed by a variety of methods. For example, mammalian uricase purity can be assessed by determining the specific activity, the number of bands which appear following electrophoresis and staining of SDS-PAGE gels, and the number and size of peaks which appear in a chromatogram following size exclusion HPLC.

### 1.2. Materials and Methods

### 1.2.1. 50 mM NaHCO₃ Buffer (pH 10.3)

This buffer was prepared by dissolving NaHCO₃ to a final concentration of 50 mM. The pH was adjusted to 10.2-10.4. Depending on starting pH, 0.1 M HCl or 1 N NaOH may be used.

### 1.2.2. 10% CPC Solution

10% CPC was prepared by dissolving CPC in distilled water to a final concentration of 10 gr/100 ml

### 1.2.3. Recombinant Porcine Uricase Expression

Recombinant mammalian uricase (urate oxidase) was expressed in E. coli K-12 strain W3110 F, as described in International Patent Publication WO00/08196 of Duke University and U.S. Patent Provisional Application No. 60/095,489, incorporated herein by reference in their entireties.

### 1.2.4. Culture and Harvest of Uricase-Producing Bacteria

Bacteria were cultured at 37° C. in growth medium containing casein hydrolysate, yeast extract, salts, glucose, and ammonia.

Following culture, bacteria in which uricase accumulated were harvested by centrifugation and washed with water to remove residual culture medium.

### 1.2.5. Cell Disruption and Recovery

Harvested cell pellet was suspended in 50 mM Tris buffer, pH 8.0 and 10 mM EDTA and brought to a final volume of approximately 20 times the dry cell weight (DCW). Lysozyme, at a concentration of 2000-3000 units/ml, was added to the suspended pellet while mixing, and incubated for 16-20 hours, at 4-8° C.

The cell lysate was treated by high shear mixing and subsequently by sonication. The suspension was diluted with an equal volume of deionized water and centrifuged. The pellet, containing uricase inclusion bodies, was diluted with deionized water (w/w) and centrifuged to further remove impurities. The pellet obtained from this last wash step was saved for further processing, and the supernatant was discarded.

### 1.2.6. Dissolution

The inclusion body (IB) pellet was suspended in 50 mM NaHCO₃ buffer, pH 10.3±0.1. The suspension was incubated at a temperature of 25±2° C. for about 0.5-2 hours to allow solubilization of the IB-derived uricase.

### 1.2.7. CPC Treatment

10% CPC solution was added in aliquots to homogenized IBs (pH 10.3), while briskly mixing, to obtain the desired CPC concentration. The sample was incubated for 1 to 24 hours as indicated, during which precipitating flakes formed. The sample was centrifuged for 15 minutes, at 12,000×g. The pellet and supernatant were separated, and the pellet was suspended with 50 mM NaHCO₃ buffer (pH 10.3) to the original volume. The enzymatic activity of each fraction was determined, and the fractions were concentrated and dialyzed to remove the remaining CPC.

### 1.2.8. Protein Assay

The protein content of aliquots of treated and untreated IB samples was determined using the modified Bradford method (Macart and Gerbaut (1982) Clin Chim Acta 122:93-101).

### 1.2.9. Uricase Assay

### 1.2.9.1. Enzymatic Activity

Activity of uricase was measured by the UV method (Fridovich, I. (1965) The competitive inhibition of uricase by oxonate and by related derivatives of s-triazines. J Biol Chem, 240, 2491-2494; modified by incorporation of 1 mg/ml BSA). Enzymatic reaction rate was determined, in duplicate samples, by measuring the decrease in absorbance at 292 nm resulting from the oxidation of uric acid to allantoin. One activity unit is defined as the quantity of uricase required to oxidize one μmole of uric acid per minute, at 25° C., at the specified conditions. Uricase potency is expressed in activity units per mg protein (U/mg).

The extinction coefficient of 1 mM uric acid at 292 nm in a 1 cm path length is 12.2. Therefore, oxidation of 1 μmole of uric acid per ml reaction mixture results in a decrease in absorbance of 12.2 mA292. The absorbance change with time (ΔA₂₉₂ per minute) was derived from the linear portion of the curve. Uricase activity was then calculated as follows:

$$Activity ( U / ml ) = \frac{Δ \text{?} ( AU / min ) \times DF \times {V}_{RM}}{{V}_{S} \times 12.2}$$

$$Where : DF = Dilution factor ;$$

$$\text{?} \text{indicates text missing or illegible when filed}$$

V_{RM}=Total volume of reaction mixture (in μl)
V_{S}=Volume of diluted sample used in reaction mixture (in μl)
   1.2.9.2. HPLC Analysis with Superdex 200

The amount and the relative percentage of the native uricase enzyme, as well as possible contaminants, were quantified according to the elution profile obtained by HPLC using a Superdex 200 column. Duplicate samples of uricase solution were injected into the column. The areas of each peak and the percent of total area were automatically calculated and summarized in the adjacent tables.

### 1.2.10. SDS-PAGE Analysis

Proteins in samples containing ˜20 □g protein/lane, were separated on 15% SDS-PAGE gels. The resulting gels were stained with Coomassie brilliant blue.

### 1.3. Results

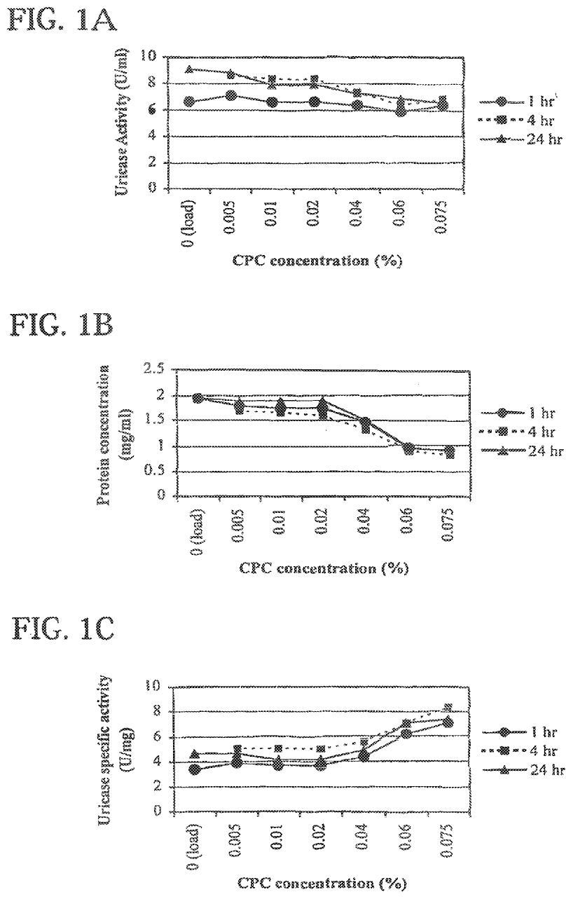

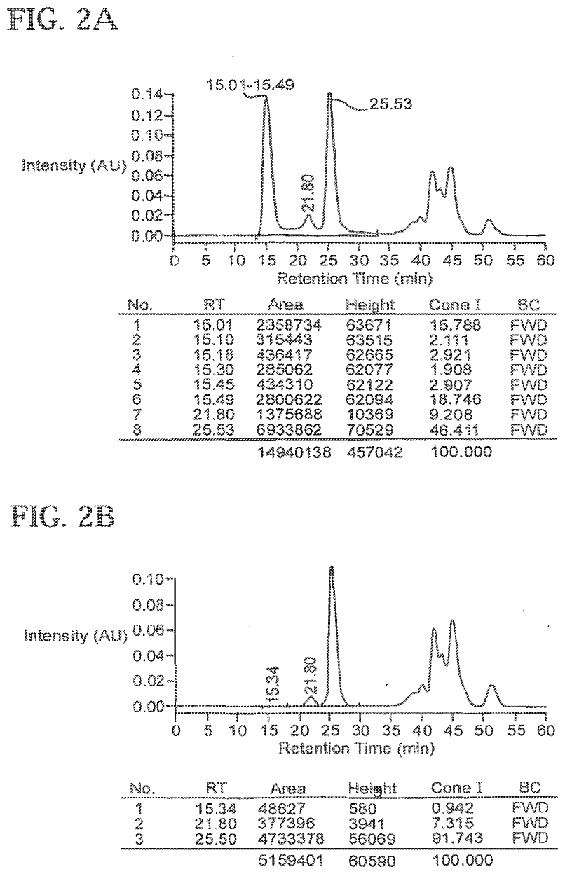

The effects of CPC (0.005-0.075%) treatment (for 1-24 hours) on uricase activity recovered in the supernatant, and its purity are presented in Table I and FIG. 1. Prior to CPC treatment (at pH 10.3), the protein concentration was 1.95 mg/ml, and the specific enzymatic activity was 3.4-4.67 U/mg. The results presented in FIG. 1B indicate that within each incubation period, the protein concentration of the supernatant decreased with increasing CPC concentration. At less than 0.04% CPC, a relatively minor effect on the protein concentration was observed. CPC, in concentrations of 0.04% to 0.075%, could reduce the protein concentration to about 50% of the original concentration.

In contrast to the effects of CPC on total protein concentration, the total soluble uricase activity was not significantly influenced by increasing CPC concentration and incubation time (FIG. 1A). Within each incubation period, the specific enzymatic activity (FIG. 1C) consistently increased as a function of CPC concentration within the range 0.04%-0.075%. This increase was a result of specific removal of non-uricase proteins. Since the specific enzymatic activity of the final purified enzyme was approximately 9 U/mg, the majority of contaminating proteins were removed by CPC precipitation. Indeed, HPLC and SDS-PAGE analyses performed support this conclusion.

TABLE 1
EFFECT OF CPC EXPOSURE ON URICASE
SPECIFIC ACTIVITY AND PURITY

| Incubation | | Uricase | | |
|---|---|---|---|---|
| time | | activity | [Protein] | Uricase specific |
| (hr) | [CPC] (%) | (U/ml) | (mg/ml) | activity (U/mg) |
| 1 | 0 (load) | 6.63 | 1.95 | 3.4 |
| 1 | 0.005 | 7.1 | 1.8 | 3.9 |
| 1 | 0.01 | 6.63 | 1.75 | 3.7 |
| 1 | 0.02 | 6.63 | 1.75 | 3.7 |
| 1 | 0.04 | 6.4 | 1.47 | 4.35 |
| 1 | 0.06 | 5.9 | 0.95 | 6.2 |
| 1 | 0.075 | 6.4 | 0.9 | 7.1 |
| 4 | 0.005 | 8.61 | 1.7 | 5.06 |
| 4 | 0.01 | 8.36 | 1.66 | 5.04 |
| 4 | 0.02 | 8.36 | 1.6 | 5.04 |
| 4 | 0.04 | 7.38 | 1.32 | 5.59 |
| 4 | 0.06 | 6.4 | 0.9 | 7.1 |
| 4 | 0.075 | 6.9 | 0.82 | 8.4 |
| 24 | 0.005 | 8.8 | 1.9 | 4.66 |
| 24 | 0.01 | 7.9 | 1.9 | 4.14 |
| 24 | 0.02 | 7.9 | 1.9 | 4.14 |
| 24 | 0.04 | 7.3 | 1.5 | 4.9 |
| 24 | 0.06 | 6.9 | 0.97 | 7.1 |
| 24 | 0.075 | 6.6 | 0.9 | 7.4 |
| 24 | 0 (load) | 9.1 | 1.95 | 4.67 |

### 1.4. Confirmation of CPC Enhancement of Uricase Purity

Uricase-containing 11Bs were isolated and solubilized, as described in section 1.3. Samples of the soluble material were analyzed prior to CPC treatment and following filtration of the CPC-precipitated protein.

1.4.1. HPLC Analysis of Non-Uricase Proteins Following Treatment with 0.075% CPC

HPLC analysis of solubilized 11Bs indicated that the uricase-associated peak (retention time (RT) ˜25.5 minutes) comprises about 46% of the protein of the crude 11B sample (FIG. 2A). Following CPC treatment, the uricase-associated peak increased to approximately 92% of the protein (FIG. 2B), and was accompanied by significant reduction of the contaminants eluting between RT 15-22 min. (FIG. 2A). The area of the uricase peak is approximately 70% of that in FIG. 2A. Thus, these results indicate a doubling of uricase purity resulting from removal of non-uricase protein upon CPC treatment.

### 1.4.2. Effect of 0.075% CPC on Enzymatic Activity

The results (presented in Table 2) indicate that mass balance of uricase activity was retained during the treatment process. CPC exposure was found to precipitate 60% of all proteins in solution. More than 85% of the enzymatic activity remained in solution, thus the removal of extraneous protein afforded an increase in specific activity of the produced supernatant of more than 110%. As in most purification processes, some of the desired activity remained in the pellet. In this instance, only 17.6% of the original activity remained in the pellet (and was extracted using 50 mM sodium bicarbonate (7 mSi, pH 10.3) for analytical purposes), which is a relatively minor fraction of the total amount.

TABLE 2
EFFECT OF CPC TREATMENT ON URICASE ACTIVITY

| | Total | | | Specific | Activity |
|---|---|---|---|---|---|
| | activity | Activity | [Protein] | activity | recovered |
| Sample | (U) | (U/ml) | (mg/ml) | (U/mg) | (%) |
| Before CPC | 490 | 4.9 | 2 | 2.46 | 100 |
| After | 418 | 4.18 | 0.8 | 5.2 | 85.3 |
| CPC | | | | | |
| treatment | | | | | |
| Pellet after CPC | 86 | 0.8 | — | — | 17.6 |
| treatment | | | | | |

1.4.3. SDS-PAGE Analysis Following Treatment with 0.075% CPC

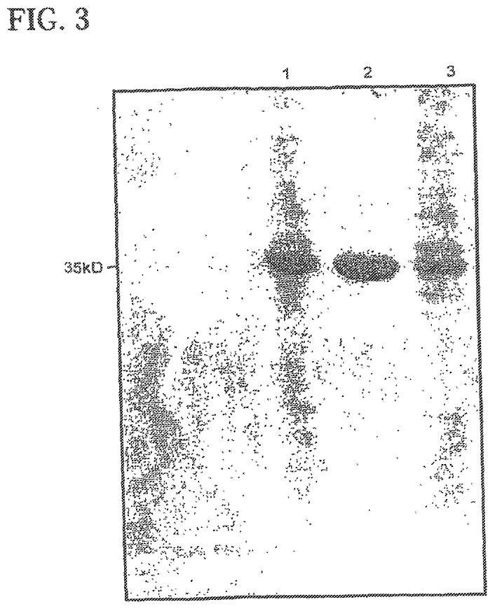

Samples of the crude uricase, prior to exposure to CPC, and of the subsequent fractions, following separation of soluble and insoluble material, following CPC treatment, centrifugal separation of the fractions, and reconstitution of the pellet obtained after centrifugation, containing equal amounts of protein were analyzed by SDS-PAGE methodology. The results (see FIG. 3) show the presence of contaminating proteins prior to CPC treatment. Following CPC treatment, the pellet contained most of the contaminating proteins, while the supernatant contained uricase that resulted in the single major protein band.

### Example 2. Effect of CPC on Purification of Single Chain (scFv) Antibodies

### 2.1. Materials and Methods

### 2.1.1. Buffers

### 2.1.1.1. Inclusion Body Dissolution Buffer

Dissolution buffer contained 6 M urea, 50 mM Tris, 1 mM EDTA, and 0.1 M cysteine.

The pH of the buffer was titrated to 8.5.

### 2.1.1.2. Folding Buffer

Folding buffer contained 1 M urea, 0.25 mM NaCl, 1 mM EDTA, and 0.1 M cysteine. The pH of the buffer was titrated to 10.0.

2.1.2. Expression of scFv Antibodies in Bacteria

ScFv antibodies (pI 8.9) were expressed in E. coli transformed with a vector encoding a scFv having cysteine-lysine-alanine-lysine at the carboxyl end as described in PCT Publication WO 02/059264, incorporated herein by reference in its entirety.

2.1.3. Culture and Harvest of scFv Antibody-Producing Bacteria

ScFv-containing bacterial cells were cultured in minimal medium, at pH 7.2, and supplemented with L-arginine, final concentration 0.5%, during the five hour period prior to induction. Expression of scFv was induced by limitation of glucose amount in the medium. ScFv-containing bacterial cells were harvested from culture by ultra filtration.

### 2.1.4. Cell Disruption and Recovery of Inclusion Bodies

Harvested cell pellet was suspended in 50 mM Tris buffer, pH 8.0 and 10 mM EDTA and brought to a final volume of approximately 20 times the dry cell weight (DCW). Lysozyme, at a concentration of 2000-3000 units/ml, was added to the suspended pellet while mixing, then incubated for 16-20 hours, at 4° C.

The cell lysate was then treated by high shear mixing and subsequently by sonication. The scFv antibody-containing inclusion bodies were recovered by centrifugation at 10,000×g. The pellet was diluted approximately sixteen fold with deionized water (w/w) and centrifuged to further remove impurities. The pellet obtained from this last wash step was saved for further processing.

### 2.1.5. Dissolution and Refolding

The IB-enriched pellet was suspended in inclusion body dissolution buffer (see above), incubated for 5 hours at room temperature, and refolded in vitro in a solution based on arginine/oxidized glutathione. After refolding, the protein was dialyzed and concentrated by tangential flow filtration against containing urea/phosphate buffer.

### 2.1.6. CPC Treatment

10^{%} CPC solution was added to the scFv refolding mixture to a final concentration of 0.02%, and after 1-2 hr incubation, at room temperature, the precipitate was removed by filtration. The supernatant contained the scFv antibody.

### 2.2. Results

2.2.1. Effect of CPC Concentration on Recoverable scFv Antibody

The effects of CPC (at pH 7.5 or 10) on scFv antibody purity and recovery are presented in Table 3. Prior to CPC treatment, the initial amount of IB protein was 73 mg, containing 15.87 mg scFv antibody as determined by HPLC analysis on Superdex 75. The retention time (RT) of the scFv antibody-containing peaks was approximately 20.6 minutes. The results indicate that recovery of total protein generally decreased with increasing CPC concentration, and recovery of scFv antibody remained >80% when the CPC concentration was <0.03%. More efficient removal of contaminating protein was achieved at pH 7.5 relative to that at pH 10. Thus, scFv antibody purification was achieved by treatment with 0.01 to 0.03% CPC.

TABLE 3
Effect of CPC treatment on scFv antibody recovery and purity

| | Total | Total scFv by | | |
|---|---|---|---|---|
| Treatment of | protein | HPLC | Purification | % recovery of |
| soluble IBs | (mg) | (mg) | factor | scFv by HPLC |
| Control (before | 73 | 15.87 | | 100 |
| CPC) | | | | |
| 0.01% CPC (pH | 64 | 15.66 | 1.13 | 98.68 |
| 10) | | | | |
| 0.01% CPC (pH | 50.76 | 14.97 | 1.36 | 94.33 |
| 7.5) | | | | |
| 0.015% CPC (pH | 54 | 14.49 | 1.23 | 91.30 |
| 10) | | | | |
| 0.015% CPC (pH | 39.96 | 14.22 | 1.64 | 89.60 |
| 7.5) | | | | |
| 0.02% CPC (pH | 43 | 13.35 | 1.43 | 84.12 |
| 10) | | | | |
| 0.02% CPC (pH | 37.8 | 13.02 | 1.58 | 82.04 |
| 7.5) | | | | |
| 0.03% CPC (pH | 35 | 11.12 | 1.46 | 70.07 |
| 10) | | | | |
| 0.03% CPC (pH | 37.8 | 12.47 | 1.52 | 78.58 |
| 7.5) | | | | |

### 2.3. Confirmation of CPC Enhancement of Scfv Antibody Purity

2.3.1. HPLC Analysis of scFv Recovery Following Treatment with CPC

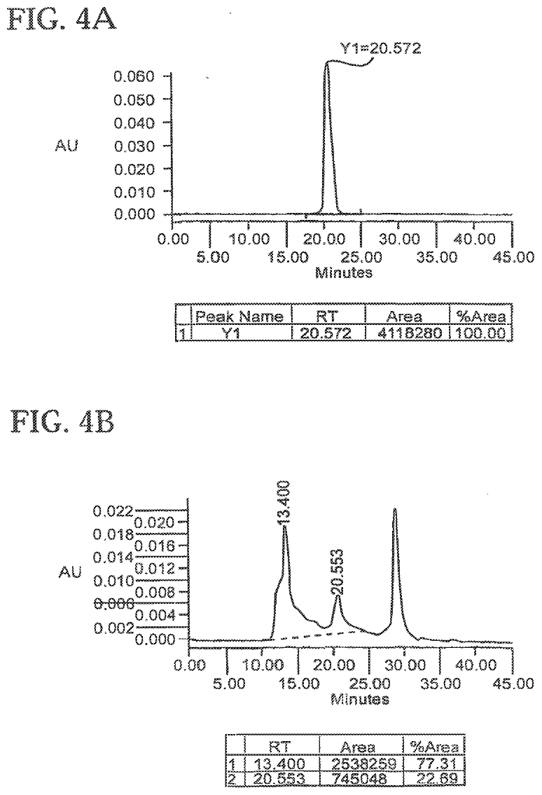

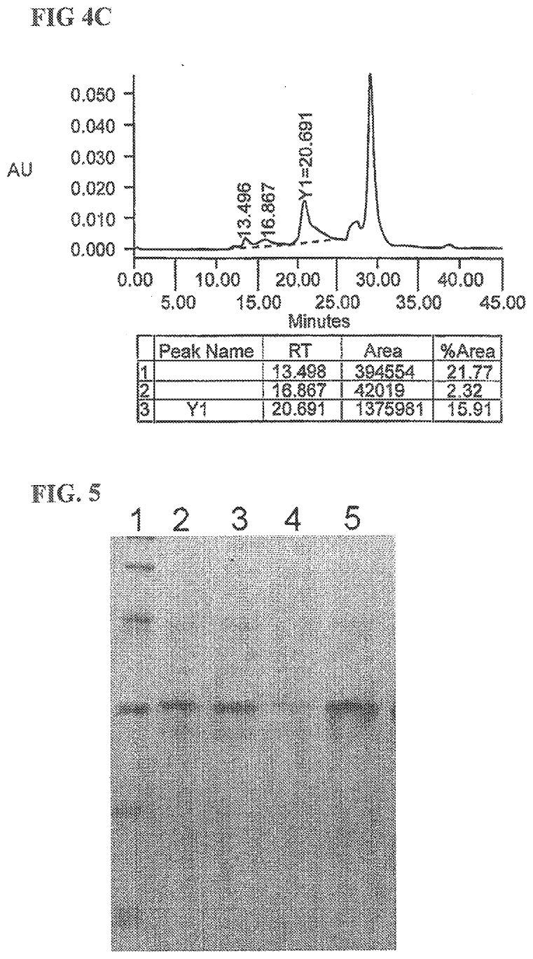

HPLC analysis of refolded protein indicates that the scFv antibody-associated peak (retention time (RT) ˜20.6 minutes) comprised about 22.7% of the protein of the total protein (FIG. 4B). The chromatogram of FIG. 4C indicates that following treatment with 0.02% CPC, the scFv antibody-associated peak of the supernatant comprised approximately 75.9% of the total protein injected, a 3.3-fold purification. Thus, CPC treatment removed protein impurities from scFv antibody solutions.

2.3.2. SDS-PAGE Analysis on scFv Recovery Following Treatment with CPC

The results (see FIG. 5) indicate that prior to CPC treatment, the sample contained significant amounts of a large number of proteins. Similarly, following CPC treatment, the pellet contained a large number of proteins. In contrast, the post-CPC treatment supernatant contained one major protein band, that of scFv antibody.

### Example 3. Effect of CPC on Purification of Recombinant Interferon-Beta

Interferon beta (IFN-beta, pI 8.5-8.9) was expressed in E-coli by known methods. Nagola, S. et al., Nature, 284:316 (1980); Goeddel, D. V. et al., Nature, 287:411 (1980); Yelverton, E. et al., Nuc. Acid Res., 9:731 (1981); Streuli, M. et al., Proc. Nat'l Acad. Sci. (U.S.), 78:2848 (1981); European Pat. Application No. 28033, published May 6, 1981; 321134, published Jul. 15, 1981; 34307 published Aug. 26, 1981; and Belgian Patent No. 837379, issued Jul. 1, 1981 described various methods for the production of beta-interferon employing recombinant DNA techniques. Procedures for recovering and purifying bacterially produced IFNs are described in U.S. Pat. Nos. 4,450,103; 4,315,852; 4,343,735; and 4,343,736; and Derynck et al., Nature (1980) 287:193-197 and Scandella and Kornberg, Biochemistry, 10:4447 (1971). Inclusion bodies containing IFN-beta were isolated and solubilized.

The resulting solution was treated with CPC. The results shown in FIG. 6 indicate a substantial decrease in the level of contaminating proteins present after CPC treatment. The actual amount of IFN-beta (area under the peak) did not change appreciably following CPC treatment.

Table 4 summarizes the effects of the CPC treatment. Total protein (Bradford) decreased by 40%, UV absorbance decreased by about 40% but the amount of IFN-beta remained unchanged.

TABLE 4

| | | | IFNb | |
|---|---|---|---|---|
| | Protein | O.D | content | |
| Sample and Treatment | (mg/ml) | A₂₈₀ | (mg/ml)^{a} | SEC Profile |
| Control (post protein | 0.51 | 1.55 | 0.069 | Peak of R.T. 13^{b} |
| folding | | | | min is 15% of |
| no CPC, 1049-31) | | | | otal area |
| Test (post protein | 0.3 | 1.0 | 0.069 | Peak of R.T. 13^{b} |
| folding and treatment | | | | min is 7.34% of |
| with 0.05% | | | | total area |
| CPC, 1049-31) | | | | |

^{a}Quantified by Vydac C4column
^{b}The SEC profile contained several peaks. The peak eluting at 13 min (R.T. 13 min) is reduced upon treatment with CPC and corresponds to the region where high molecular weight proteins and variants thereof elute.

### Example 4. Effect of CPC on Purification of Factor Xa Inhibitor

CPC was used to purify leech factor Xa inhibitor. Leech factor Xa inhibitor (FXaI, pI 8.4-9.1) may be produced as described in U.S. Pat. No. 6,211,341 and International Patent Publication No. WO94/23735. Following isolation of FXaI-containing inclusion bodies (IBs), the FXaI was purified from IBs substantially as described in example 1. After dissolution of the IB pellet, the preparation was incubated with 10% CPC solution. Then, the mixture was centrifuged for 15 minutes, at 12,000×g. The pellet and supernatant were separated. The pellet was suspended with 50 mM NaHCO₃ buffer to the original volume. The pellet and supernatant were separately concentrated and dialyzed to remove the remaining CPC. The protein content and activity were assayed and FXaI was found to be the predominant component in the supernatant and substantially absent from the pellet. The results indicate that CPC treatment enhanced the efficiency of recovery and the purity of the recovered FXaI.

### Example 5. Purification of Carboxypeptidase B (CPB) by CPC

Identical amounts of inclusion bodies obtained from a clone expressing CPB were solubilized in 8 M urea, pH 9.5 (control and test). Production of CPB is described in International Patent Publication No. WO96/23064 and in U.S. Pat. No. 5,948,668. The test sample was treated with CPC 0.11% and clarified by filtration prior to refolding. Refolding of control and test samples were carried out by diluting the solutions 1:8 into refolding buffer. After treatment with endoproteinase over night at ambient temperature, equal amounts of control and test solutions were loaded onto a DEAE Sepharose column. The column was washed and the active enzyme was subsequently eluted with 60 mM Sodium Chloride in 20 mM Tris buffer pH 8.

TABLE 5

| | | Treatment | |
|---|---|---|---|
| Process Step | Parameter | Control | 0.11% CPC |
| Dissolution in 8M Urea | Total A₂₈₀ | 960 | 494 |
| Post Clarification | Protein Content | 490 | 272 |
| | (mg)* | | |
| | pH | 9.5 | 9.5 |
| | Enzyme Activity | Inactive | Inactive |
| | (Units) | ( ** ) | (**) |
| Post Chromatography | Protein Content | 5.67 | 8.41 |
| of 26.5 mg of Refoldate | (mg)* | | |
| (DEAE MP) | Enzyme Activity | 258 | 4043 |
| | (Units) | | |
| | Specific Activity | 98 | 481 |
| | (Units/mg) | | |

*Protein determination was carried out by the Bradford method.
**Prior to refolding the protein was inactive

The results presented in Table 5 show that total OD in the CPC treated material dropped by 49.5% and the total protein content was reduced by 44.5%. Interestingly, total enzyme activity recovered in the CPC treated sample increased by 79%, suggesting that CPC removed a component that partially inhibited generation of active enzyme.

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A method for purifying a target protein comprising identifying a target protein and contacting a solution comprising the solubilized target protein and one or more solubilized contaminating proteins with one or more cationic surfactants in an amount effective to selectively precipitate the one or more contaminating proteins.

2. The method of claim 1, further comprising the step of recovering the solubilized target protein.

3. The method of claim 1 wherein at least one of the one or more cationic surfactants is an amphipathic ammonium compound.

4. The method of claim 3 wherein the amphipathic ammonium compound is selected from the group consisting of quaternary ammonium compounds of the general formula QN⁺; paraffin chain primary ammonium compounds of the general formula RNH₃⁺; and salts thereof.

5. The method of claim 4 wherein the amphipathic ammonium compound is selected from the group consisting of cetyl pyridinium salts, stearamide-methylpyridinium salts, lauryl pyridinium salts, cetyl quinolynium salts, lauryl aminopropionic acid methyl ester salts, lauryl amino propionic acid metal salts, lauryl dimethyl betaine, stearyl dimethyl betaine, lauryl dihydroxyethyl betaine and benzethonium salts.

6. The method of claim 5 wherein the amphipathic ammonium compound is selected from hexadecylpyridinium chloride, dequalinium acetate, hexadecylpyridinium chloride, cetyltrimethylammonium chloride, mixed n-alkyl dimethyl benzylammonium chloride, cetylpyridinium chloride, N,N-dimethyl-N-[2-[2-[4-(1,1,3,3,-tetramethylbutyl)-phenoxy]ethoxy]ethyl] benzenemethanammonium chloride, alkyl-dimethylbenzyl-ammonium chloride, and dichloro-benzyldimethyl-alkylammonium chloride, tetradecyl trimethylammonium bromide, dodecyl trimethylammonium bromide, cetyl trimethylammonium bromide, lauryl dimethyl betaine stearyl dimethyl betaine, and lauryl dihydroxyethyl betaine.

7. The method of claim 5 wherein the amphipathic ammonium compound is a cetylpyridinium salt.

8. The method of claim 7 wherein the cetyl pyridinium salt is a halide salt.

9. The method of claim 8 wherein the cetyl pyridinium halide salt is cetylpyridinium chloride.

10. The method of claim 9 wherein the amphipathic ammonium compound has at least one aliphatic chain having 6-20 carbon atoms.

11. The method of claim 10 wherein the aliphatic chain has 8-18 carbon atoms.

12. The method of claim 1 wherein the solution further comprises one or more cellular components.

13. The method of claim 12 wherein the one or more cellular components are derived from a microorganism.

14. The method of claim 13 wherein the microorganism is a bacteria.

15. The method of claim 14 wherein the bacteria is E. coli.

16. The method of claim 12 wherein the one or more cellular components are one or more proteins.

17. The method of claim 1 wherein the target protein is a recombinant protein.

18. The method of claim 17 wherein the recombinant protein is an enzyme.

19. The method of claim 17 wherein the target protein is selected from the group consisting of an antibody, a uricase, an interferon-beta, a factor X inhibitor, an acid deoxyribonuclease II, an elastase, a lysozyme, a papain, a peroxidase, a pancreatic ribonuclease, a trypsinogen, a trypsin, a cytochrome c, an erabutoxin, Staphylococcus aureus enterotoxin C1, an interferon and a monoamine oxidase A.

20. The method of claim 19 wherein the target protein is a uricase.

21. The method of claim 20 wherein the uricase is a mammalian uricase.

22. The method of claim 21 wherein the mammalian uricase is a porcine uricase.

23. The method of claim 17 wherein the target protein is an antibody.

24. The method of claim 23 wherein the antibody is a single chain antibody.

25. The method of claim 17 wherein the target protein is an interferon.

26. The method of claim 25 wherein the interferon is an interferon beta.

27. The method of claim 1 wherein the one or more cationic surfactants are added to a concentration of from 0.001% to 5.0%.

28. The method of claim 27 wherein the one or more cationic surfactants are added to a concentration of from 0.01% to 0.5%.

29. The method of claim 27 wherein the one or more cationic surfactants are added to a concentration of from 0.03% to 0.2%.

30. The method of claim 1, wherein the contacting is done for from 5 minutes to 48 hours.

31. The method of claim 30, wherein the contacting is done from 10 minutes to 24 hours.

32. The method of claim 1, wherein the contacting is done at a temperature of from 4° C. to 36° C.

33. The method of claim 32, wherein the contacting is done at a temperature of from 4° C. to 26° C.

34. The method of claim 1, wherein the solution is substantially free of polyanions.

35. The method of claim 1, wherein the solution is substantially free of solid supports.

36. The method of claim 1, wherein the solution is substantially free of aggregates of the contaminating proteins with other molecules.

37. The method of claim 1, wherein the solution is substantially free of polyanions; solid supports and aggregates of the contaminating proteins with other molecules.

38. The method of claim 35, 36 or 37, wherein the cationic surfactant is a cetylpyridinium salt.

39. The method of claim 37, 38 or 39, wherein the cetylpyridinium salt is cetylpyridinium chloride.

40. The method of claim 1, wherein the target protein has an isoelectric point greater than or equal to 7.

41. A purified protein prepared according to the method of claim 1.

42. A purified uricase prepared according to the method of claim 1.

43. The uricase of claim 42 wherein the uricase is a mammalian uricase.

44. The uricase of claim 43 wherein the mammalian uricase is a porcine uricase.

45. The uricase of claim 42, wherein the uricase is from a bacterial cell, wherein the bacterial cell comprises DNA encoding the uricase and the DNA is expressed to produce the uricase.

46. The uricase of claim 45 wherein the uricase is recovered from inclusion bodies produced by the bacterial cell.

47. A method for purifying a target protein comprising the steps of:
a. identifying a target protein;
b. contacting a solution comprising solubilized target protein and one or more
c. solubilized contaminating proteins with one or more cationic surfactants in an amount effective to selectively precipitate the one or more contaminating proteins; and
d. recovering the soluble target protein.

48. A method of increasing the percentage of a target protein in a solution of proteins comprising the steps of
a. obtaining a solution of a plurality of proteins, wherein the proteins in solution comprise the target protein and contaminating proteins, and the target protein comprises a first percentage by weight of the total protein in the solution;
b. contacting the solution with one or more cationic surfactants in an amount effective to selectively precipitate the contaminating proteins;
wherein the target protein in the solution of step b comprises a second percentage by weight of the total protein, and the second percentage is greater than the first percentage.
